(19) United States
(12) Patent Application Publication
(10) Pub. No.: US 2004/0170646 A1
(43) Pub. Date: Sep. 2, 2004

(54) Production of ALVAC on avian embryonic stem cells

(75) Inventors: Veronique Barban, Craponne (FR); Luc Aujame, Fleurieux sur l'Arbresle (FR)

Correspondence Address: Patrick J. Halloran, Aventis Pasteur, Knerr Building, Swiftwater, PA, 18370, US

(73) Assignee: Aventis Pasteur, Inc., Swiftwater, PA

(21) Appl. No.: 10/735,064

(22) Filed: Dec. 12, 2003

Related U.S. Application Data
(60) Provisional application No. 60/433,332, filed on Dec. 13, 2002.

(51) Int. Cl. A61K 39/12; C12N 7/00

(52) U.S. Cl. 424/199.1; 435/235.1

(57) ABSTRACT

The present invention relates to methods for producing ALVAC virus on avian embryonic stem cells and compositions comprising ALVAC virus made using such methods.

### RELATED APPLICATIONS

This application claims priority to U.S. Prov. Appln. Ser. No. 60/433,332 filed Dec. 13, 2002.

### FIELD OF THE INVENTION

The present invention relates to improved processes for the production of ALVAC viruses using avian embryonic stem cells.

### BACKGROUND OF THE INVENTION

Current process of production of ALVAC vaccines on chicken embryo fibroblasts (CEFs) involves handling hundreds of embryonated eggs. After embryo dissociation, the cells are seeded in roller bottles before infection. Typically, about 200 eggs are needed for infection of 120 roller bottles. The use of a continuous cell line growing in suspension would allow to suppress handling of eggs and to replace roller bottles by a 20-liter biofermentor. After optimization of culture conditions, one can expect to increase the cell density, and, consequently the final viral yields. One suitable cell line that could be used for such purposes would be a stable chicken embryo fibroblast derived cell line that grows in suspension.

Avian embryonic cell lines have been generated by several different investigators. For example, Pettite, et al. (North Carolina State Univ.; U.S. Pat. No. 5,340,740) relates to the development of avian embryonic stem cells by culturing avian blastodermal cells in the presence of a mouse fibroblast feeder layer. Pettite (U.S. Pat. No. 5,656,479; WO 93/23528) also describes and claims an avian cell culture of undifferentiated avian cells expressing an embryonic stem cell phenotype.

Samarut, et al. (Institut National de la Recherche Agronomique, et al.; U.S. Pat. No. 6,114,168; WO 96/12793) describes methods for producing avian embryonic stem cells on CEFs using particular media. Bouquet, et al. (Institut National de la Recherche Agronomique; U.S. Pat. No. 6,280,970 B1; patent application No. 2001/0036656 A1, published Nov. 1, 2001) describes transformed avian embryonic fibroblasts that contain SV40 T Ag within their genome. Samarut and Pain (patent application No. 2001/0019840 A1, pub. Sep. 6, 2001) relates to culture medium for producing avian ES cells and methods for producing proteins in ES cells cultured in such medium. And, Han, et al. (Hanmi Pharm. Co. Ltd.; WO 00/47717) describes the processes for developing avian embryonic germ cell lines by culturing avian primordial germ cells in culture medium containing particular growth factors and differentiation inhibitory factors.

Avian embryonic stem cells have been shown to be suitable for producing recombinant viruses. For example, Foster, et al. (Regents of Univ. Minnesota, U.S. Pat. Nos. 5,672,485; 5,879,924; 5,985,642; 5,879,924) describes methods for growing viruses in stable cell lines derived from chicken embryo fibroblasts.

Reilly, et al. (Board of Trustees operating Michigan State University; U.S. Pat. No. 5,989,805; WO 99/24068) relates to the use of chicken embryonic stem cells modified with a chemical mutagen to produce Marek's virus, swine influenza virus, equine influenza virus, avian influenza virus, avian reovirus, folwpox virus, pigeon pox, canarypox, psittacine herpesvirus, pigeon herpesvirus, falcon herpesvirus, Newcastle disease virus, infectious bursal disease virus, infectious bronchitis virus, avian encephalomyelitis virus, chicken anemia virus, avian adenovirus, and avian polyomavirus. Coussens, et al. (Board of Trustees operating Michigan State University; U.S. Pat. Nos. 5,827,738; 5,833,980) also relates to propagation of Marek's disease virus in embryonic stem cells. Bouquet, et al. (Institut National de la Recherche Agronomique; U.S. Pat. No. 6,280,970 B1; patent application No. 2001/0036656 A1, published Nov. 1, 2001) describes methods for producing viruses from avian embryonic fibroblasts transformed by incoporation of the SV40 T Ag within their genome.

There is a need in the art for improved processes for producing ALVAC-based vaccines. Provided herein is one such method that provides for production of ALVAC vectors using avian embryonic stem cell lines growing in suspension. The method provides both production and safety advantages. The significant aspects of the present invention are described below.

### SUMMARY OF THE INVENTION

The present invention provides methods for propagating ALVAC viruses, preparing vaccines and providing vaccines to hosts by culturing an ALVAC virus in avian embryonic stem cells and harvesting the virus from the cells. Preferred cells are EB1 or EB14 cells. In certain embodiments, the virus has within its genome exogenous DNA encoding an immunogen that, upon expression within a host to whom the virus has been administered, results in a protective immune response.

### BRIEF DESCRIPTION OF THE DRAWINGS

FIG. 1. Progressive adaptation of cells to DMEM/F12 medium.

FIG. 2. Cell culture analysis for Test 1.

FIG. 3. Additional cell culture analysis for Test 1.

FIG. 4. EB1 infection with vCP205

### DETAILED DESCRIPTION

The present application provides novel methods for culturing ALVAC viruses on embryonic stem cells. All references cited within this application are incorporated by reference.

Poxvirus is a useful expression vector (Smith, et al. 1983, Gene, 25 (1): 21-8; Moss, et al, 1992, Biotechnology, 20: 345-62; Moss, et al, 1992, Curr. Top. Microbiol. Immunol., 158: 25-38; Moss, et al. 1991. Science, 252: 1662-1667). The canarypox ALVAC is a particularly useful virus for expressing exogenous DNA sequences in host cells. ALVAC-based recombinant viruses (i.e., ALVAC-1 and ALVAC-2) are particularly suitable in practicing the present invention (see, for. example, U.S. Pat. No. 5,756,103). ALVAC(2) is identical to ALVAC(1) except that ALVAC(2) genome comprises the vaccinia E3L and K3L genes under the control of vaccinia promoters (U.S. Pat. No. 6,130,066; Beattie et al., 1995a, 1995b, 1991; Chang et al., 1992; Davies et al., 1993). Both ALVAC(1) and ALVAC(2) have been demonstrated to be useful in expressing foreign DNA sequences, such as TAs (Tartaglia et al., 1993a,b; U.S. Pat. No. 5,833,975). ALVAC was deposited under the terms of the Budapest Treaty with the American Type Culture Collection (ATCC), 10801 University Boulevard, Manassas, Va. 20110-2209, USA, ATCC accession number VR-2547.

ALVAC has been demonstrated to be useful for expressing exogenous DNA sequences in host cells (see, for example, U.S. Pat. Nos. 5,756,102; 5,833,975; 5,843,456; 5,858,373; 5,863,542; 5,942,235; 5,989,561; 5,997,878; 6,265,189; 6,267,965; 6,309,647; 6,541,458; 6,596,279; and, 6,632,438). In practicing the present invention, ALVAC may be cultured in its native state or as a recombinant containing an exogenous DNA encoding a protein such as an antigen. Particularly useful antigens would include those derived from pathogens that cause disease in humans (i.e., a human pathogen) such as a bacterium, fungus, or virus, among others, or antigens derived from tumors (i.e., tumor or tumor-associated antigens). Many such antigens are known in the art and would be suitable in practicing the present invention. The ALVAC vector may also encode immune co-stimulatory molecules such as B7.1, among others. The invention further includes compositions containing ALVAC vectors in pharmaceutically acceptable diluents. The administration of such compositions to animal or human hosts in need of immunization is also contemplated.

In one embodiment, the present invention demonstrates that it is possible to produce ALVAC virus, on continuous, non-tumorigenic avian cells derived from avian embryonic stem cells. Suitable cells for such purposes have been described in, for example, U.S. Pat. Nos. 5,340,740; 5,656,479; 5,672,485; 5,879,924; 5,985,642; 5,989,805; 6,114,168; 6,280,970 B1; U.S. patent application Nos. 2001/0036656 A1; 2001/0019840 A1; and, international applications WO 93/23528; WO 96/12793; WO 99/24068; WO 00/47717; FR02/02945; and WO 03/07661). In certain embodiments, such cells include, for example, EB1, EB2, EB3, EB4, EB5, and EB14 cells (as described in FR02/02945 and WO 03/07661). These cells were obtained from chick embryos at very early steps of embryogenesis and exhibit a stem cell phenotype. The cells are not genetically modified in their native state and grow in suspension. In one embodiment, the cells are EB1 cells obtained from VIVALIS SA (France; FR02/02945 and WO 03/07661). In a second embodiment, the cells are EB14 cells obtained from VIVALIS SA (FR02/02945 and WO 03/07661). EB1 and EB14 cells are an early expansion of avian embryonic stem cells. Suitable cells such as these are included within the definition of the term “avian embryonic stem cell line” (“AES”). Any of such cells, along with other AES that are known in the art, may be suitable in practicing the present invention.

A better understanding of the present invention and of its many advantages will be had from the following examples, given by way of illustration.

### EXAMPLES

### Example 1

### Material and Methods

A. Cells and virus

EB1 cells (2×50×10^{E}6 cells) were received at p139 (May 2001) or p148 (July 2001) from Vivalis. The culture medium (Modified McCoy 5% and 0% SVF), was provided with the cells. All infections were performed using ALVAC vCP205 (ATCC No. VR-2557; U.S. Pat. No. 5,863,542; HIV expression cassette—vaccinia H6 promoter/HIV truncated env MN strain, I3L gag with protease in ALVAC C3 insertion site), #362, clarified (titer 7.9 logTCID50/ml), purified (sucrose cushion+gradient, titer 8.5 log TCID50/ml), or semi-purified (sucrose cushion, titer 9.2 logTCID50).

The genealogy of EB1 cells is shown below:

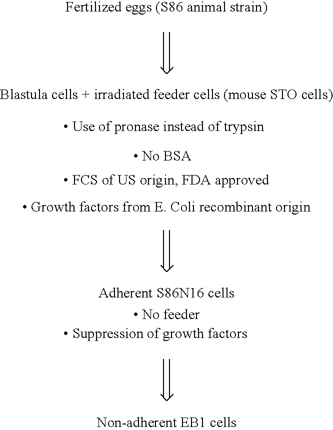

B. Processing of Infected Cells

Infected cells were harvested by centrifugation. Cell pellets were resuspended in 1/20 to 1/20 of the initial volume of the culture medium without serum, sonicated briefly in culture medium and centrifuged again to obtain the clarified lysate.

C. Viral Quantification

In order to study ALVAC DNA replication in viral preparations, we developed an ALVAC DNA quantitative PCR assay with the LightCycler™ apparatus. ALVAC DNA was purified and amplified in presence of SYBR Green Dye using primers specific for KIOR region, encoding structural VP8 protein. A standard curve, established from known concentrations of purified viral DNA, was used to estimate the viral DNA concentration in each sample. ALVAC DNA was quantified by QPCR on LightCycler, following SOP V100501/01 as described below:

A. Equipment: L2 class zone; Type II flow laminar hoods in 2 separated rooms with 2 different colors coats; LightCycler with a carousel (Roche Diagnistics Ref:2011468); capillaries (Roche Diagnostics ref: 1909339); centrifuge adapters (Roche Diagnostics ref:1909312); centrifuge (Eppendorf Ref:5415D); carousel centrifuge (Roche Diagnostics Ref:2189682); box with ice; thin wall 96 well plate model M (COSTAR Ref:6511); micro test tube, 1.5 ml (Eppendorf Ref:24077); 8 channel electronic pipette, 0.2-10 μl (BIOHIT ref:710200); barrier tips 10, 20, 50, 200, 1000 μl; and, 10, 50, 200, 1000 μl manual pipettes.

B. Products: ALVAC standard DNA, 5 tenfold dilutions: 20 to 200,000 copies; internal reference for extraction and quantification: ALVAC virus, 10⁷ TCID50/ml (about 2×10⁹ copies/ml); FastStart DNA Master SYBR Green I kit ((Roche Diagnostics ref:2239264); H₂O, DNase and RNase free (PROMEGA Ref: P1193); samples: ALVAC DNA or ALVAC virus; primers CPK1011 (5 μM) and CPK1012 (5 μM) (see below):

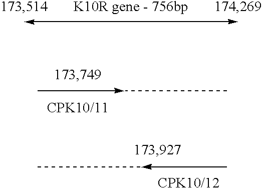

C. Precautions: wear gloves; Master Mix and DNA dilutions must be performed in 2 different hoods; SYBR Green must be protected from light and conserved at 5° C.±1° C.; Adapters must be pre-cooled at 5° C.±1° C. in the cooling block.

D. Procedure:

Start Lightcycler: Before sample preparation, using the LightCycler software, select the program (FastStart 50° C.) and define the number of samples, and label properly.

Prepare master mix preparation (on ice):

Prepare the reaction mix under the first hood, on ice. Use 1.5 ml reaction tubes, and calculate the volume needed for 5 standard points, 1 negative point, 1 reference point and n+1 samples.

Add 60 μl of 1b tube to 1a tube. Mix by pipetting (do not vortex).

| Products | [Final] | Volume (μl) |
|---|---|---|
| H₂O (Promega) | | 11.6 |
| MgCl₂ | 4 mM | 2.4 |
| CPK1011/CPK1012 | 0.5 μM/0.5 μM | 2 |
| SYBR Green mix | 1× | 2 |

Put 18 μl of mix in each capillary. The cooling block is then transferred under the second hood.

DNA preparation:

On ice, dilute ALVAC DNA samples with DNase/Rnase-free H₂O in micro tubes or in 96 well plate, in order to have less than 200,000 copies (estimated) by capillary.

Dilute ALVAC DNA standard from 200,000 to 20 copies (tenfold dilutions).

Dilute ALVAC reference DNA 100 fold.

In each capillary, add 2 μl of DNA template, or 2 μl of H₂O in the negative sample. Seal the capillary with a plastic stopper. Centrifuge the adapters (which contain the capillaries) 30 sec in a centrifuge at 100 g and put the capillaries into the carousel. Place the carousel containing the samples in the LightCycler and close the lid.

Start the run.

Analyzed by LightCycler software

For quantification select analysis method:

Chose “Fits Points method”

Step 1 :chose “arithmetic base line”

Select standard samples

Step 2: adjust the noise band to eliminate the fluorescence background.

Step 3: adjust the cross line so that the error value is lower than 0.1, with a slope value between −3.3 and −4.0 (optimal theoretical value 3.4) and an intercept value between 30 and 40. At the optimal setting for the line, the calculated values of the standard should be closest to their known values.

For Tm analysis select melting curve analysis:

Step 1: select “linear with background” method

Select samples

Step 2: adjust the cursors at the beginning and at the end of the melting pea, respectively.

Step 3: select “manual Tm”: the software calculates the Tm for the sample.

Controls

Baseline fluorescence values should be close to zero for all samples

Two parameters allow validation of the standard curve. The first one is the error that should be below 0.1. The second one is the second-degree equation, with a slope value comprised between −3.3 and −4.0 (optimal theoretical value 3.4) and an intercept value between 30 and 40.

The melting curve of the PCR product allows to control the specificity of primers: Tm value is usually about 78±1° C. Specificity can also be controlled on agarose gel electrophoresis: only one product should be amplified, at 110 bp.

The internal reference is used to control the quality of DNA extraction.

Infectious titers were measured by a standard PFU assay.

### Example 2

### Growth Optimization for EB1 Cells

Prior to use, the cells were analyzed to optimize conditions for growth. As described above, EB1 cells were provided by VIVALIS in the specific modified medium McCoy-5% FCS. The influence of two parameters FCS (2.5% versus 5%) and C02 (0% versus 5%) on EB1 cell growth has been tested. Adaptation of the cells to DMEM-F12 medium has also been tested. For each condition, the generation time was calculated.

To carry out the tests, spinners were inoculated at an initial concentration of 10⁴ cells/ml in the chosen conditions and incubated at 37° C. under agitation. As soon as the medium became acidic, cells were diluted to a concentration of 10⁴ to 10⁵/ml in fresh medium. Cell viability was measured by Trypan blue exclusion. In each instance in which cell viability was too low (i.e.<70%), a Ficoll gradient was performed to eliminate dead cells (indicated by arrows A and C on the graphs).

Progressive adaptation of cells to DMEM/F12 medium was accomplished by progressively diluting the initial medium (McCoy medium) with DMEM/F12 (indicated by arrow C on the graph). Generation time (G) corresponds to the number of doublings (or generations) per day, and is calculated according to G=N/D, where D is the number of days of culture and N is the number of generations determined from the equation C_{f}=Cᵢ×2^{N}, C_{f} and Cᵢ being respectively the final and initial cell concentrations.

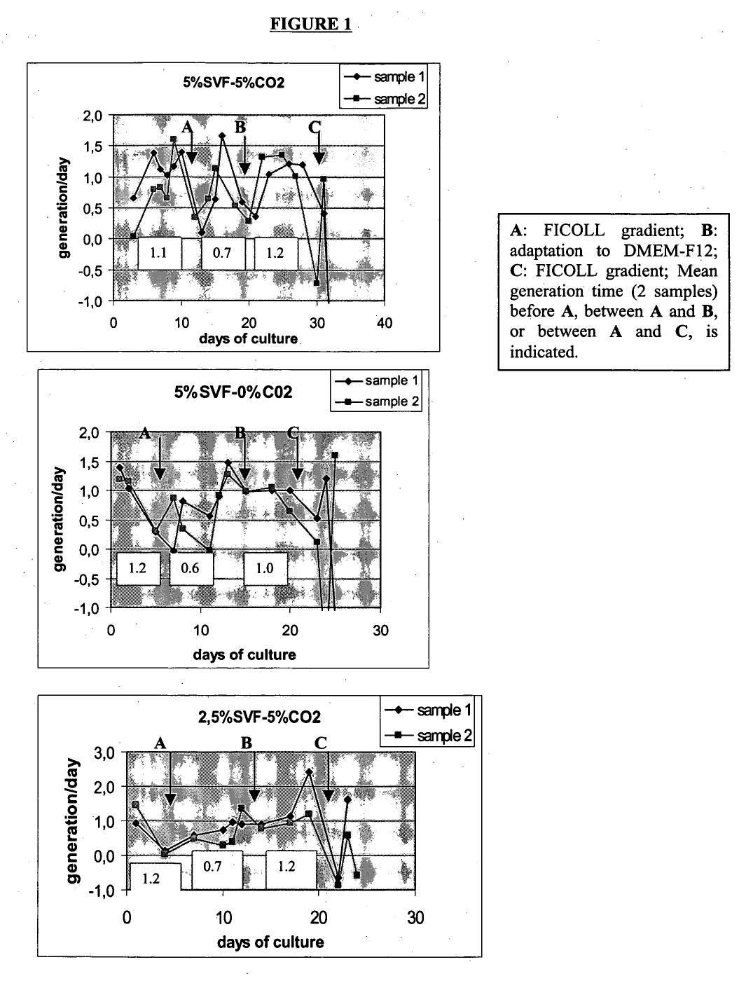

The data has been obtained by cell numeration of non-infected cells, and presented as a function of initial density of cells. The results of these studies are summarized in FIG. 1 and Table 1.

TABLE 1

| Initial cell density | culture days | | |
|---|---|---|---|
| Cells/ml (× 1000) | 1 | 2 | 3 |
| 4-20 | 1.09 +/− 0.42 | 1.24 +/− 0.61 | nd |
| 20-100 | 1.4 +/− 0.14 | 1.05 +/− 0.21 | 1.18 +/− 0.17 |
| 100-500 | 1.15 +/− 0.27 | nd | 0.19 +/− 0.14 |

From these studies, it has been concluded that:

The mean doubling time of EB1 cells in suspension is about 1.1 generation/day;

There is no significant difference in growth curves when cells are cultivated in presence of 2.5 or 5% FCS.

The cells are sensitive to Ficoll gradient centrifugation, and conditions should be optimized.

The maximal density of cells we have reached in our conditions is about 800,000 cells/ml. At higher density, culture medium becomes acid, cell growth is stopped, cells undergo apoptosis and degenerate rapidly.

EB1 cells can be grown as suspensions in standard DMEM-F12 medium containing 2.5% FCS, with an average doubling time of about 1 generation per day.

The maximum cell density in spinner is between 5×10⁵ and 10⁶ cells/ml, but culture conditions in a biogenerator may be useful for increasing the biomass.

### Example 3

### Infection of EB1 Cells in Spinner

A. Test 1

100 ml of EB1 cells (P138) in DMEM-F12-0% FCS (initial density: 4×10⁵ cells/ml) were incubated for 1 h at 37° C. with a clarified preparation of ALVAC-HIV vCP205 (m.o.i 0.1). The culture was then diluted with an equivalent volume of modified McCOY5A-5% FCS (final cell density: 2×10⁵ cells/ml), and incubated at 37° C. under agitation (spinner) and 5% CO₂. Both cell fraction and culture fluid were collected at 48 and 96 hours p.i., and analyzed for infectious virus (PFU assay on CEPs) and viral DNA content (qPCR). At each time point, 20 ml of the culture were analyzed. After centrifugation, the supernatant fraction (S) was collected and directly used for quantification. The pellet, corresponding to the cell fraction (C) was re-suspended in 1 ml (1:20 of initial volume) of Tris 10 mM pH9, before sonication and quantification. The titers are expressed per ml (left column) or per fraction (right column). The total viral material produced in the spinner was calculated by adding the 2 fractions: Total=(S/ml×200)+(C/ml×10). The total value per ml was obtained by dividing this result by 200. The results of this test are shown in Table 2.

TABLE 2

| | |
|---|---|
| spinner 48 h | spinner 96 h |

| | /ml | /fraction | /ml | /fraction |
|---|---|---|---|---|
| Log GEQ | | | | |
| cell fraction | 6.25* | 7.55 | 5.76* | 7.07 |
| supernatant | 4.75 | 7.04 | 6.42 | 8.72 |
| Total | 5.37 | 7.67 | 6.43 | 8.73 |

| | | |
|---|---|---|
| GEQ/cell | 1.2 | 13.4 |

| | | | | |
|---|---|---|---|---|
| Log PFU | | | | |
| cell fraction | 4.95* | 6.25 | 4.94* | 6.25 |
| supernatant | 4.30 | 6.60 | 6.26 | 8.56 |
| Total | 4.45 | 6.75 | 6.27 | 8.57 |

| | | |
|---|---|---|
| PFU/cell | 0.14 | 9.3 |

*titer estimated after concentration of cells in 1:20 of initial volume

B. Test 2

22.5 ml of cells (P138) in suspension in DMEM-F12-0% FCS (initial density: 5.6×10⁵ cells/ml) were incubated for 30 min. at 37° C. with a clarified preparation of ALVAC-HIV vCP205 (m.o.i 0.1). The culture was then diluted with an equivalent volume of modified McCOY5A-5% FCS (final cell density: 2.8×10⁵ cells/ml), and incubated at 37° C. under agitation (spinner) and 5% CO₂. Both cell fraction and culture fluid were collected at 50, 74 and 96 hours p.i., and analyzed for infectious virus (PFU assay) and viral DNA content (qPCR). Cell culture analysis was performed as described for Test 1, above. Results of this test are summarized in Table 3.

TABLE 3

| | 50 hours | | 74 hours | | 97 hours | |
|---|---|---|---|---|---|---|
| | /ml | /fraction | /ml | /fraction | /ml | /fraction |
| Log GEQ | | | | | | |
| Cell fraction | 6.89* | 7.54 | 7.15* | 7.80 | 7.31* | 7.97 |
| supernatant | 6.05 | 7.70 | 6.54 | 8.20 | 6.96 | 8.61 |
| total | 6.28 | 7.93 | 6.69 | 8.35 | 7.05 | 8.70 |
| GEQ/cell | 30.4 | | 80 | | 179 | |
| log PFU | | | | | | |
| Cell fraction | 6.40* | 7.05 | 6.37* | 7.02 | 5.99* | 6.64 |
| supernatant | 5.56 | 7.21 | 5.8 | 7.45 | 6.29 | 7.94 |
| total | 5.78 | 7.44 | 5.94 | 7.60 | 6.31 | 7.96 |

| | | | |
|---|---|---|---|
| PFU/cell | 2.2 | 3.2 | 7.2 |

*titer estimated after concentration of cells in 1:5 of initial volume

C. Test 3

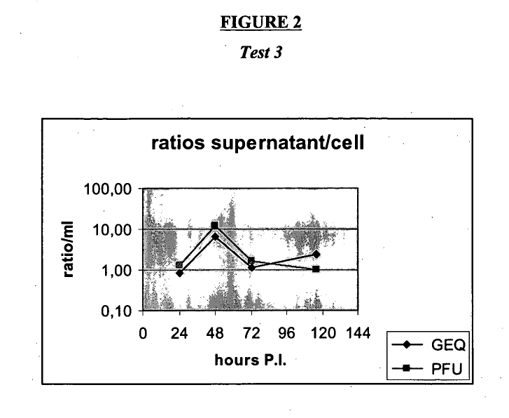

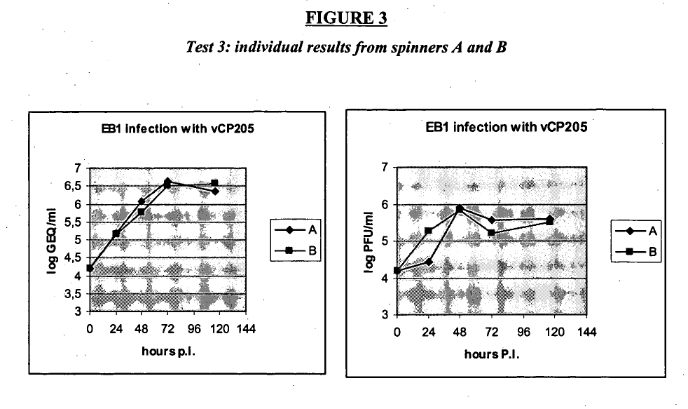

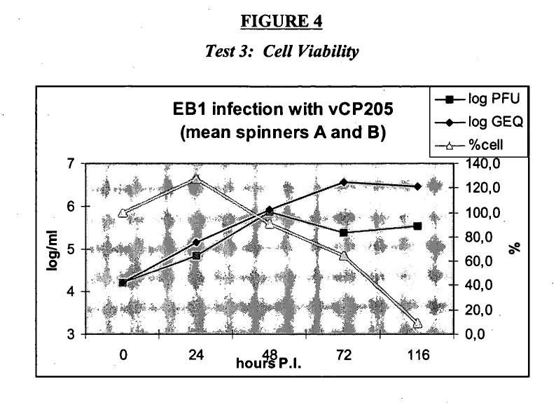

EB1 cells at p148 were infected in a minimal volume (5 ml) of modified McCOY 5A medium −0%FCS at an m.o.i. of 0.1, and diluted at a final density of 1.5×10⁵ cells/ml in 200 ml of modified McCoy medium 2% FCS. The experiment was done in duplicate (spinners A and B), cells were infected with semi-purified (sucrose cushion, spinner A) or purified (sucrose cushion+gradient, spinner B) preparations of vCP205 (#363). Both viral DNA and infectious virus were quantified in the cell fraction and in the supernatant of infected cells at time-points 24, 48, 72 and 116 h. P.I. No significant differences were obtained between spinner A and spinner B. Cell culture analysis was performed as described for Test 1, above. Results of this test are summarized in Tables 4 and 5 as well as FIGS. 2 and 3. Cell viability was also measured in parallel, as shown in FIG. 4.

TABLE 4
× 10E6 cells/ml

| | cell number | | | % cells | | |
|---|---|---|---|---|---|---|
| hours p.i. | A | B | Mean | A | B | mean |
| 0 | 31.4 | 31.4 | 31.4 | 100.0 | 100.0 | 100.0 |
| 24 | 38 | 42 | 40 | 121.0 | 133.8 | 127.4 |
| 48 | 30.2 | 27 | 28.6 | 96.2 | 86.0 | 91.1 |
| 72 | 20 | 20.6 | 20.3 | 63.7 | 65.6 | 64.6 |
| 116 | 2.75 | 2.75 | 2.75 | 8.8 | 8.8 | 8.8 |

| 24 | | 8 | | 72 | | 116 | |
|---|---|---|---|---|---|---|---|
| | /frac- | | /frac- | | /frac- | | /frac- |
| /ml | tion | /ml | tion | /ml | tion | /ml | tion |

spinner A

| | | | | | | | | |
|---|---|---|---|---|---|---|---|---|
| log GEQ | | | | | | | | |
| cell fraction | 5.93 | 7.23 | 5.97 | 7.27 | 7.28 | 8.58 | 6.89 | 8.19 |
| supernatant | 4.8 | 7.1 | 6.03 | 8.33 | 6.39 | 8.69 | 6.18 | 8.48 |
| GEQ total | 5.17 | 7.47 | 6.07 | 8.27 | 6.64 | 8.94 | 6.36 | 8.66 |
| GEQ/cell | | 0.9 | | 7.4 | | 27.7 | | 14.6 |
| log PFU | | | | | | | | |
| cell fraction | 5.9 | 7.2 | 5.7 | 7 | 6.13 | 7.43 | 6.60 | 7.9 |
| supernatant | 4.4 | 6.73 | 5.9 | 8.21 | 5.6 | 7.86 | 5.60 | 7.89 |
| PFU total | 5.43 | 6.73 | 5.91 | 8.21 | 5.56 | 7.86 | 5.59 | 7.89 |
| PFU/cell | | 0.2 | | 5.2 | | 2.3 | | 2.5 |

spinner B

| | | | | | | | | |
|---|---|---|---|---|---|---|---|---|
| log GEQ | | | | | | | | |
| cell fraction | 5.86 | 7.16 | 6.07 | 7.38 | 7.19 | 8.49 | 6.99 | 8.29 |
| supernatant | 4.82 | 7.12 | 5.67 | 7.97 | 6.21 | 8.51 | 6.43 | 8.73 |
| GEQ total | 5.14 | 7.44 | 5.77 | 8.07 | 6.50 | 8.80 | 6.56 | 8.66 |
| GEQ/cell | | 0.9 | | 3.7 | | 20.1 | | 23.3 |
| log PFU | | | | | | | | |
| cell fraction | 5.56 | 6.86 | 5.91 | 7.21 | 6.19 | 7.49 | 6.50 | 7.8 |
| supernatant | 5.3 | 7.56 | 5.84 | 8.14 | 5.2 | 7.5 | 5.50 | 7.81 |
| PFU total | 4.26 | 7.56 | 5.84 | 8.14 | 5.20 | 7.50 | 5.51 | 7.81 |
| PFU/cell | | 1.2 | | 4.4 | | 1.0 | | 2.1 |

Mean ratios supernatant/cell associated viruses (spinner A and B)
Ratio = [PFU/GEQ medium]/[PFU/GEQ cell fraction]

TABLE 5
mean values spinners [A, B]/ml

| | 24 h | | 48 h | | 72 h | | 116 h | |
|---|---|---|---|---|---|---|---|---|
| | | /frac- | | /frac- | | /frac- | | /frac- |
| | /ml | tion | /ml | tion | /ml | tion | /ml | tion |
| Log GEQ | | | | | | | | |
| cell fraction | 5.90* | 7.20 | 6.02* | 7.33 | 7.24* | 8.54 | 6.94* | 8.24 |
| supernatant | 4.81 | 7.11 | 5.85 | 8.15 | 6.30 | 8.60 | 6.31 | 8.61 |
| GEQ total | 5.16 | 7.46 | 5.92 | 8.22 | 6.57 | 8.87 | 6.46 | 8.76 |
| GEQ/cell | 0.91 | | 5.6 | | 24 | | 19 | |
| log PFU | | | | | | | | |
| cell fraction | 5.73* | 7.03 | 5.81* | 7.11 | 6.16* | 7.46 | 6.55* | 7.85 |
| supernatant | 4.85 | 7.15 | 5.87 | 8.18 | 5.40 | 7.68 | 5.55 | 7.85 |
| PFU total | 4.84 | 7.15 | 5.87 | 8.18 | 5.38 | 7.68 | 5.55 | 7.85 |

| | | | | |
|---|---|---|---|---|
| PFU/cell | 0.4 | 4.8 | 1.5 | 2.3 |

*titers estimated after concentration of cells to 1:20 of initial volume

D. Infections in Static Conditions, Without Agitation (Flasks)

75 cm culture flasks were seeded with 3×10⁶ cells in a total volume of 50 ml of DMEM-F12 without FCS, and infected with vCP205 at an m.o.i. of 0.1 for 48 hours at 37° C., under 5% C0₂. Culture fluids and cell fractions were collected and infectious virus (PFU assay) and viral DNA (qPCR) were quantified. The results of this test are summarized in Table 6 and FIG. 4.

TABLE 6

| | F75 n°1 | | F75 n°2 | | F75 n°3 | | F75 n°4 | |
|---|---|---|---|---|---|---|---|---|
| | | /frac- | | /frac- | | /frac- | | /frac- |
| | /ml | tion | /ml | tion | /ml | tion | /ml | tion |
| Log GEQ | | | | | | | | |
| cell fraction | 6.41* | 6.41 | 6.37* | 6.37 | 6.43* | 6.43 | 6.37* | 6.37 |
| supernatant | 6.24 | 7.94 | 6.28 | 7.97 | 6.26 | 7.95 | 6.25 | 7.94 |
| total | 6.25 | 7.95 | 6.28 | 7.98 | 6.26 | 7.96 | 6.25 | 7.95 |
| GEQ/cell | 30 | | 32 | | 30 | | 30 | |
| Log PFU | | | | | | | | |
| cell fraction | 4.37* | 4.37 | 4.31* | 4.31 | 4.43* | 4.43 | 4.52* | 4.52 |
| supernatant | 4.45 | 6.15 | 4.61 | 6.31 | 4.43 | 6.13 | 4.33 | 6.03 |
| total | 4.46 | 6.16 | 4.61 | 6.31 | 4.44 | 6.14 | 4.34 | 6.04 |

| | | | | |
|---|---|---|---|---|
| PFU/cell | 0.5 | 0.7 | 0.5 | 0.4 |

*titer estimated after concentration of cells in 1 ml (1:50 of initial volume)

The following conclusions have been reached from this study:

Viral yields are higher when cells are cultivated in spinners instead of flasks (mean value: 5 PFU/ml versus 0.5 PFU/ml);

Mean PFU titer/cell: 6.3 (vs 2.5 TCID50/cell for CEPs grown virus as determined from the mean value calculated from vCP205 #S3317, #S3292, #3124, #LST011 and #LP012);

Mean GEQ titer per cell: 105 (vs125 GEQ/cell for CEPs grown vCP205). As a comparison, the viral yield in chick embryo fibroblasts (CEPs) is routinely about 2.5 TCID₅₀/cell (5 to 20 PFU), corresponding to 125 GEQ/cell;

In McCoy Medium: DMEM/F12 (1:1) 2.5% FCS, maximal titer (both infectious and genomic) is reached between 72 and 97 hours p.i. In McCoy Medium 2.5% FCS, genomic titer increases until 116 h. p.i., while infectious titer is stable at 48 h.p.i.;

in Tests 1 and 2, the virus is mainly recovered from the cell culture supernatant, which is most likely a consequence of cell lysis;

EB1 cells replicate ALVAC vCP205 at similar yields than CEPs; and,

With no optimization, based on a viral yield of 6 PFU/cell and a cell density of 5×10⁵ cells/ml, a standard production process of 120 roller bottles could be replaced by one 20-liter biogenerator.

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. A method for propagating an ALVAC virus, comprising: (a) infecting one or mores avian embryonic stem cells with an ALVAC virus; (b) cultivating the infected avian embryonic stem cells to produce the virus; and (c) isolating the virus.

2. The method of any one of claims 1 wherein the virus comprises an exogenous DNA sequence within the ALVAC genome.

3. The method of claim 2 wherein the exogenous DNA encodes a tumor antigen, an antigen derived from a human pathogen, or a fragment thereof.

4. The method of claim 3 wherein the pathogen is bacterial, fungal or viral.

5. A method selected from the group consisting of a method of claims 1, 2, 3 and 4, wherein the exogenous DNA further encodes a co-stimulatory molecule.

6. The method of claim 5 wherein the exogenous DNA encodes the co-stimulatory molecule B7.1.

7. A method for propagating a virus, comprising: (a) infecting one or more cells derived from an avian embryonic stem cell with an ALVAC virus; (b) cultivating the infected cells to produce the virus; and, (c) isolating the virus.

8. The method of claim 7 wherein the cells are EB1 or EB14 cells.

9. The method of claim 7 or 8 wherein the virus comprises an exogenous DNA sequence within the ALVAC genome.

10. The method of claim 9 wherein the exogenous DNA encodes a tumor antigen, an antigen derived from a human pathogen, or a fragment thereof.

11. The method of claim 10 wherein the pathogen is bacterial, fungal or viral.

12. The method of claim 11 wherein the exogenous DNA further encodes a co-stimulatory molecule.

13. The method of claim 12 wherein the exogenous DNA encodes the co-stimulatory molecule B7.1.

14. A method selected from the group consisting of a method of claims 1, 2, 3, 4, 5, 6, 7, 8, 9, 10, 11, 12 and 13, wherein the ALVAC virus is ALVAC(2).

15. A composition comprising an ALVAC virus produced by a method selected from the group consisting of the methods of claims 1, 2, 3, 4, 5, 6, 7, 8, 9, 10, 11, 12, 13, and 14.

16. A method for preparing a immunogenic composition comprising: (a) infecting avian embryonic stem cells with an ALVAC virus comprising within the ALVAC genome at least one exogenous nucleotide sequence encoding a human tumor antigen, an antigen derived from a human pathogen, or a fragment thereof; (b) cultivating the infected cells to produce the virus; (c) harvesting the virus from the cultivated cells; and, (d) subjecting the harvested virus to at least one of the following treatments: (i) inactivating the virus, (ii) adding a pharmaceutically acceptable carrier or diluent, (iii) adding an adjuvant, or (iv) lyophilization.

17. The method of claim 17 wherein the ALVAC virus is ALVAC(2).

18. A composition comprising an ALVAC virus produced by the method of claim 17 or 18.

19. A method for preparing a immunogenic composition comprising: (a) infecting one or more cells derived from an avian embryonic stem cell with an ALVAC virus comprising within the ALVAC genome at least one exogenous nucleotide sequence encoding an antigen derived from a human tumor antigen, an antigen derived from a human pathogen, or a fragment thereof; (b) cultivating the infected cells to produce the virus; (c) harvesting the virus from the cultivated cells; and, (d) subjecting the harvested virus to at least one of the following treatments: (i) inactivating the virus, (ii) adding a pharmaceutically acceptable carrier or diluent, (iii) adding an adjuvant, or (iv) lyophilization.

20. The method of claim 19 wherein the ALVAC virus is ALVAC(2).

21. The method of claim 19 or 20 wherein the cells are EB1 or EB14 cells.

22. A composition comprising an ALVAC virus produced by a method selected from the group consisting of the methods of claims 19, 20, and 21.

23. A composition useful in the manufacture of a medicament for the treatment of human disease, the composition comprising an ALVAC virus produced by a method selected from the group consisting of the methods of claims 19, 20, 21, and 22.

24. A method for providing a vaccine to a host, comprising: (a) infecting avian embryonic stem cells with an ALVAC virus having within the ALVAC genome at least one exogenous nucleotide sequence encoding a human tumor antigen, an antigen derived from a human pathogen, or a fragment thereof; (b) cultivating the infected cells to produce the virus; (c) harvesting the virus from the cultivated cells; (d) subjecting the harvested virus to at least one of the following treatments: (i) inactivating the virus, (ii) adding a pharmaceutically acceptable carrier or diluent, (iii) adding an adjuvant, (iv) adding a stabilizer, or (v) lyophilizing to produce a vaccinal composition; and, (e) administering the vaccinal composition to the host whereby a protective immune response occurs in the host.

25. The method of claim 24 wherein the cells are EB1 or EB14 cells.

26. The method of claim 24 or 25 wherein the ALVAC virus is ALVAC(2).

27. A composition comprising an ALVAC virus produced by a method selected from the group consisting of the methods of claims 24, 25, and 26.

28. A composition useful in the manufacture of a medicament for the treatment of human disease, the composition comprising an ALVAC virus produced by a method selected from the group consisting of the methods of claims 24, 25, 26, and 27.
